(19) United States
(12) Patent Application Publication
(10) Pub. No.: US 2006/0255191 A1
(43) Pub. Date: Nov. 16, 2006

(54) Reciprocating autoclave with internal cutters (RAIC) and method for treatment of medical waste

(76) Inventors: Chow-Feng Chiang, Taichung City (TW); Mao-Hsiang Lin, Taichung City (TW)

Correspondence Address: HDSL, 4331 STEVENS BATTLE LANE, FAIRFAX, VA, 22033, US

(21) Appl. No.: 11/126,318

(22) Filed: May 11, 2005

(51) Int. Cl. A61L 2/07; B02C 19/00

(52) U.S. Cl. 241/23; 422/26; 422/309

(57) ABSTRACT

An apparatus and method for treatment of medical waste materials, namely a reciprocating autoclave with internal cutters (RAIC), capable of simultaneous crushing and sterilizing of the waste within a waste-treating vessel under a desired temperature and pressure by passing the waste back and forth through the cutters at free falling of the waste materials during the reciprocating of the vessel. It enhances heat penetration onto the small pieces of the fully fractured waste for highly efficient sterilization.

### BACKGROUND OF THE INVENTION

1. Field of the Invention

This invention is concerning an apparatus and method for treatment of medical waste materials, and in particular, to a reciprocating autoclave with internal cutters (RAIC), capable of simultaneously crushing and sterilizing of the waste materials to avoid secondary contamination, characterized in that the entire treatment can be completed at reduced time, comparing with conventional autoclaving methods. The invention is particularly designed to eliminate aerosol pollution during the pre-vacuum operation by employing a separate gas buffering and cooling vessel for pressure regulation. The medical waste can be satisfactorily transformed (decoded) into regular wastes for final treatment and disposal.

2. Description of the Prior Art

Comparing with regular solid waste treatment, the treatment of medical wastes has been very costly and laborious, primarily due to the pathogenic hazards and public perception associated with the waste. The waste must be satisfactorily processed to comply with many strict regulations imposed for pollution control as well as for public perception. Accordingly, the treatment of medical wastes requires consideration of the following requirements:

(1) The combustible medical waste is segregated and stored in red bag and non-combustible in yellow bag for easy identification and the followed-up treatment (color management).

(2) The wastes should be completely sterilized to destroy pathogens.

(3) A strain of thermophilic bacteria, such as Bacillus stearothermophilus spores, is normally used as the indicating organism. The concentrations of the spore before and after treatment (C1 and C2) are analyzed and a sterilization efficiency is computed as:
Log (kill)=Log(C1/C2)

in which, C1 and C2 are expressed as colony forming unit per milliliter of test suspension (cfu/mL). Most countries require a value of Log (kill) not exceeding 5 for an acceptable degree of pathogen destruction. This is also called a “five-of-nine” treatment or a treatment efficiency of 99.999%.

(4) The waste should not be recognized by visual inspection from appearance as the medical waste after satisfactory treatment (unrecognizability), such as for the body tissue, syringe, needle, glass bottle, and tubing, etc.

(5) It must be well controlled during treatment of the wastes to avoid the possible pollution, such as odor, air, and wastewater. Otherwise, effective pollution control devices must be equipped.

To our best knowledge, none of the existing technologies can completely achieve all the requirements stated above. The technology cited in U.S. Pat. No. 5,119,994 “Method and Apparatus for the Treatment of Medical Waste Materials” was evaluated against these requirements. The cited apparatus is basically an airtight vessel into which wastes are charged. Air is withdrawn out to a certain degree of vacuum and hot steam is injected into the vessel to a certain pressure and temperature for sterilization. A helically configured member and lifting paddles are installed along the interior perimeter of the vessel to assist on mixing of the waste during the treatment. After completion, the waste is discharged out of the vessel for crushing into small pieces by a cutting device installed outside the vessel (post crushing). The broken materials are then treated or disposed by landfill or incineration as for the regular domestic wastes. However the cited technique is bearing with the following shortages:

1. The cited method requires the separation of metals and rubbers from the waste before treatment to prevent the post-shredder from malfunction.

2. The cited method uses activated carbon to adsorb bioaerosols emitted from the vessel for pre-vacuum operation. The contaminated activated carbon requires further treatment and disposal.

3. The vessel is installed along the interior perimeter with a helically configured member and attached lifting paddles. Although having advantageous mixing of waste materials, the helical member provides little crushing, which is critical to complete steam penetration to the waste materials for efficient sterilization.

4. The period of time required for the cited technology should be as long as for the conventional methods (normally 80 minutes) for complete sterilization.

5. After sterilization, the cited method does not offer a mean to cool off the waste. If not waiting for sufficient time before opening the vessel, steamy vapor will be emitted out of the vessel and cause possible damage or interference to the operators.

6. The waste being treated in the vessel requires a separate crushing to make it unrecognizable.

7. The post-shredding device requires more installation space and laborious handling work.

In light of the numerous shortcomings stated above, a technology is invented by incorporating a multiple-shaft cutter device within a waste-treating vessel to achieve better steam penetration for sterilization of the medical wastes. In conjunction with the waste-treating vessel, a separate gas buffering and cooling vessel is facilitated for the purpose of complete sterilization of the contaminated air from the pre-vacuum operation. A novel operating procedure is also proposed to take the advantage of this novel apparatus to avoid laborious sorting and possible secondary pollution during the treatment of the medical wastes.

### SUMMARY OF THE INVENTION

This invention is to provide a multiple-shaft crushing device installed inside a reciprocating vessel for the treatment of medical wastes. This new design will allow for the entire bag of wastes to be fed into the vessel without unnecessary sorting (intact feeding), so that possible infection from personal contact to the wastes can be avoided.

The main consideration of this invention is to facilitate an internal crushing device for simultaneous sterilization and crushing of the waste. The steam penetration to the wastes can be significantly improved for efficient sterilization under a desired pressure and temperature. The volume reduction of the wastes can also be efficiently improved.

Another, this invention is to facilitate a novel operating procedure accompanying with the new device, so that the wastes can be processed with proper sequences of treatment cycles to avoid unnecessary secondary pollution.

This invention first facilitates a waste-treating vessel having a volume adequate for the design capacity. The vessel is divided into a feed chamber, an output chamber and an inventory chamber. While allowing for a batch feed of the design capacity, the feed chamber must be sufficiently large to maintain a filling ratio less than 70˜80% of the total volume of the vessel. The volume of the storage chamber must be adequate for holding the waste materials during crushing, especially the initial stage of the treatment. The fractured waste is moving back and forth across the cutter device between the feed chamber and the storage chamber at free falling of the wastes by reciprocating the entire vessel.

Instead of using a conventional air pollution control device, such as the activated carbon, this invention facilitates a separate gas buffering and cooling vessel, preferably installed as a jacket enveloping the waste-treating vessel for the convenience of piping arrangement. The air in the gas buffering and cooling vessel is first withdrawn to the atmosphere until a preset degree of vacuum is achieved. The waste-treating vessel is then loaded with the medical wastes. The air in the waste-treating vessel is then withdrawn from the loaded vessel to the pre-vacuum gas buffering and cooling vessel until a preset degree of vacuum in the waste-treating vessel is achieved. This is important to prevent the waste-treating vessel from cold spot when the steam is introduced into the vessel for sterilization.

The valve between the gas buffering and cooling vessel and the waste-treating vessel is then closed and the steam is injected into the waste-treating vessel until a preset pressure is reached. The cutter device and the driving system are turned on to begin the crushing of the waste materials under a preset temperature and pressure for sterilization. After a period of time, the valve between the gas buffering and cooling vessel and the waste-treating vessel is turned on to allow for circulation of the hot steam between the two vessels, so that the contaminated air originally stored in the gas buffering and cooling vessel is sterilized.

The cutter and transmission system comprises at least 2 sets of multiple-shaft cutter devices, each being configured on a plate base, one responsible for the shredding of wastes into larger pieces and another for grinding into smaller pieces. Each set of cutters is installed on a plate base. The two sets of cutter bases are positioned to each other at a vertical angle with respect to orientation of the cutter shafts to accomplish a highly efficient crushing of the wastes. The simultaneous crushing and sterilization is important for efficient steam penetration onto all the fractured pieces of the waste materials. The entire operation can be accomplished in a much shorter period of time (30 minutes) with a higher volume reduction (15˜20%), comparing with other conventional methods.

After completing the treatment, the cutter and transmission system are tuned off. The valve of steam supply is turned off and the heat exchange system installed in the gas buffering and cooling vessel is turned on to cool off the steam and lower down the pressure in the waste-treating vessel. The waste-treating vessel is then rotated to a position and the inlet closure member is opened to discharge the fully processed waste materials. The waste is now ready for further treatment or disposal as for the regular domestic waste (refuse).

### BRIEF DESCRIPTION OF THE DRAWINGS

The drawings disclose the illustrative embodiments of the present invention which serves to exemplify the various advantages and objects hereof, and are as follows:

FIG. 1 is a side elevation view of the novel apparatus, used for simultaneous sterilization and crushing of medical waste, showing the waste-treating vessel and the reciprocating wheel, such vessel containing an internal cutter and transmission system and an external buffering jacket, also showing the cutter and transmission system;

FIG. 1a is a cross sectional view taken along line in FIG. 1, showing two sets of cutter and transmission systems positioned at a 90-degree angle to the orientation to the shafts of the cutter base;

FIG. 2 is a side view of the novel apparatus, showing the waste-treating vessel 1, the buffering jacket and, all the related piping system 4 for pressure regulation, this view revealing the heat exchange system with the heat exchange pipeline configured inside the buffering jacket;

FIG. 2a is a cross sectional view taken along line 2a in FIG. 2;

FIG. 3 is a cross sectional view illustrating two sets of the cutter and transmission systems 2;

FIG. 3a is a top view of one set of the cutter and transmission system 2a, illustrating the cutter base, cutter, and cutter shaft;

FIG. 3b is a top view of another set of the cutter and transmission system, illustrating the cutter base, cutter, and cutter shaft;

FIG. 4 is a side view of the supporting base 75 for the novel apparatus, showing the driving motor and driving gear;

FIG. 4a is a side view of FIG. 4, showing the driving motor and driving gear;

FIG. 5 is a side view of the reciprocating wheel;

FIG. 5a is a side view of FIG. 5, illustrating the reciprocating wheel;

FIG. 5b is another side view of FIG. 5, illustrating the enlarged wheel chain/gear; and

FIG. 6 is a flow chart of the operation procedure of this invention.

### DETAILED DESCRIPTION OF THE PREFERRED EMBODIMENTS

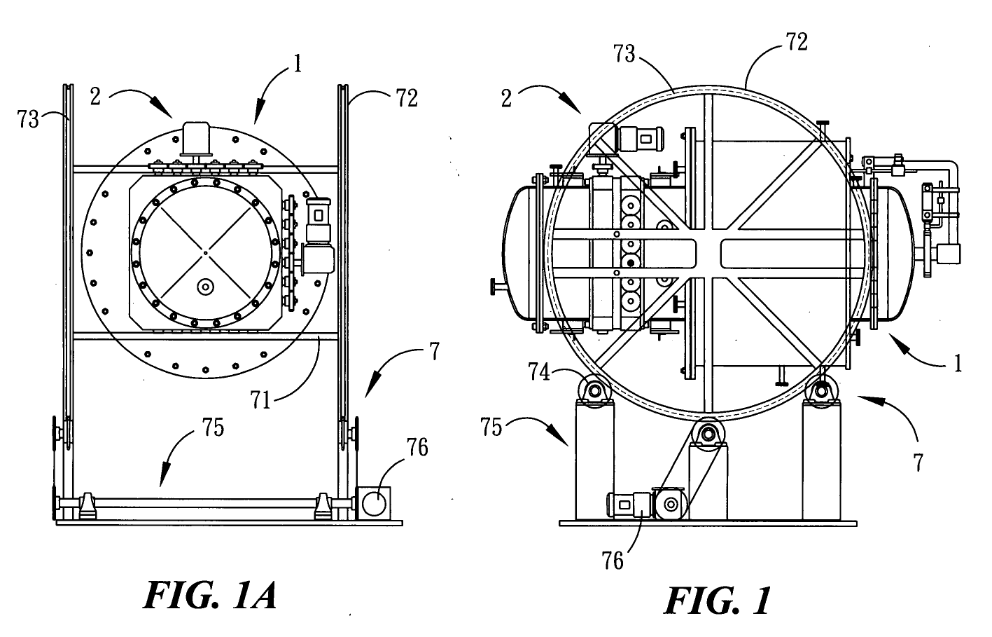

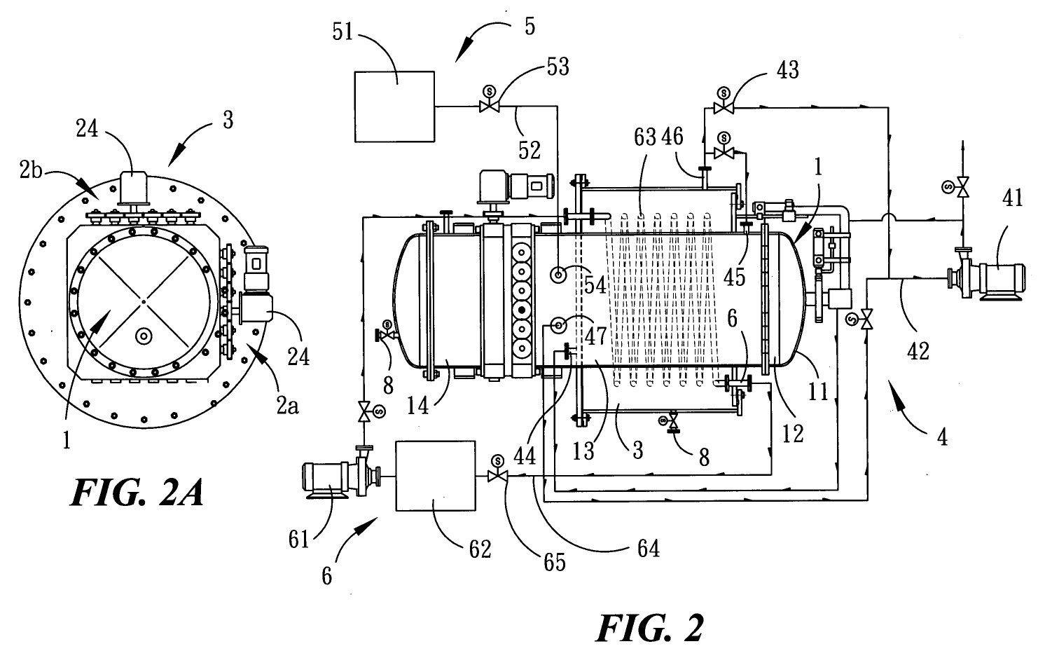

As shown in FIG. 1 to 2a, this invention facilitates a novel internal multiple-shaft crushing apparatus for the treatment of medical wastes. The major parts include: one set of waste-treating vessel 1, two sets of cutter devices 2, one set of gas buffering and cooling vessel 3, one set of vacuum system 4, one set of steam supply system 5, one set of heat exchange system 6, and one set of driving system for the vessel 7. The use of this apparatus also involves a novel method.

This invention includes a waste-treating vessel 1 for the containing and treatment of medical wastes (not shown in the diagram). The wall of the waste-treating vessel 1 is manufactured with a vacuum layer for better insulation. Located at the front end of the vessel is a feed inlet and outlet 12 for waste feeding and removal, and a closure member 11 is provided covering the feed inlet and outlet 12, by which the waste-treating vessel 1 can be closely sealed during the treatment of wastes. Inside the waste-treating vessel 1 and extended from the feed inlet and outlet 12 is a space of feed charging and discharging chamber 13, while extended from the feed charging and discharging chamber 13 to the rear end of the vessel is the space of a storage chamber 14. The volume of the feed charging and discharging chamber 13 is designed to accommodate the batch treatment capacity and normally takes up 70˜75% of the entire volume of the waste-treating vessel 1 to comply with the regulation on the feed charging ratio not more than 75-80%.

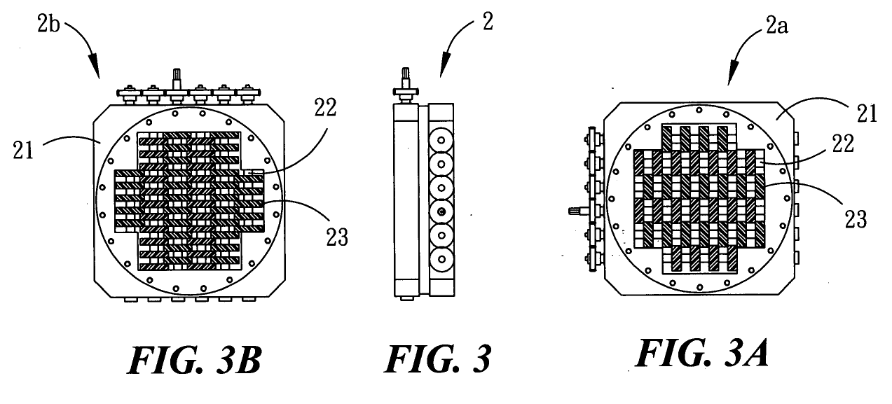

With reference of FIG. 3˜3b, a cutter and transmission system 2 is located between the feed charging and discharging chamber 13 and the storage chamber 14 inside the vessel 1 for reciprocating cutting of the medical waste. The cutter and transmission system comprises a set of cutters 23 which are mounted on a set of cutter shafts 22. The cutter shafts 22 are mounted apart from each other with an equal spacing on a base plate 21. On each pair of shafts 22, each cutter 23 is positioned with an equal spacing along the shaft so that each cutter 23 on one shaft 22 is alternatively docked into the spacing between two cutters on the neighboring shafts. The spacing between two shafts 22 is properly arranged to avoid possible clogging during treatment. All the cutters 23 and shafts 22 are powered by a motor 24 and transmission systems 2, 2a, and 2b in a manner that cutters 23 on adjacent shafts 22 engage with one another to give a function of crushing the medical waste. Furthermore, in order to achieve better crushing effect, two or more sets of cutters can be installed in the cutter and transmission system 2, one for coarse crushing (shredding) with a larger shaft spacing and another for fine crushing (grinding) with a smaller shaft spacing. The shredding cutter is normally placed near the feed charging and discharging chamber 13 and the grinding cutter near the storage chamber 14. In addition, the two cutter plates are positioned so that the orientation of their shafts is arranged at an angle of 90 degrees to avoid possible dead zone of crushing. By this novel arrangement of cutter and transmission system 2, 2a, and 2b, medical waste materials are effectively fractured into small pieces and completely processed into regular wastes.

As shown in FIG. 2, the gas buffering and cooling vessel 3 is installed outside the waste-treating vessel 1 to cool down the internal temperature of the waste-treating vessel 1 and to envelop the entire feed charging and discharging chamber 13 for the convenience of piping arrangement and better insulation.

The air circulation system 4 comprises a set of vacuum pump 41, two set of pipeline 42, and four sets of in-line electromagnetic valves 43. The feed charging and discharging chamber 13 is connected by one pipeline of the circulation system 4 with an inlet 45 and an outlet 47. The gas buffering and cooling vessel 3 is also connected by another pipeline of the circulation system 4 with an inlet 44 and an outlet 46. As shown in FIG. 2, the two types of pipeline are also interchangeably connected for the convenience of air pressure regulation and circulation.

To begin with the operation for the gas buffering and cooling vessel 3, close the inlet 44 while keeping the outlet 46 open. Turn on the vacuum pump 41 to withdraw air from the gas buffering and cooling vessel 3 to the atmosphere until a preset vacuum is achieved. After closing the outlet 46 of the gas buffering and cooling vessel, turn on the inlet 44 of the gas buffering and cooling vessel and the outlet 47 of the waste-treating vessel. Turn on the vacuum pump 41 to withdraw air from the waste-treating vessel 1 to the gas buffering and cooling vessel 3 until a preset vacuum is achieved. Turn off the outlet 47 of the waste-treating vessel 1 and the inlet 44 of the gas buffering and cooling vessel 3.

The steam supply system 5 consists of a steam generator 51, a steam pipeline 52, and an in-line steam electromagnetic valve 53. The steam pipeline 52 is connected into the waste-treating vessel 1 with a steam inlet 54. Turn on the steam inlet 54 and the steam generator 51 to supply steam into the waste-treating vessel 1 until a preset pressure and temperature is achieved.

The heat exchange system 6 consists of a cooling water circulation pump 61, a cooling device 62, a heat exchanger 63, a cooling water pipeline 64 and a cooling water electromagnetic valve 65 at the cooling water pipeline 64, in which the heat exchanger 63 is installed within the gas buffering and cooling vessel 3 and on the outside surface of the waste-treating vessel 1. After completion of the sterilization, turn on the cooling water pump so that the cooling water is circulated through the cooling water pipeline 64 into the heat exchanger 63 to cool the hot steam in the waste-treating vessel 1. The water condensed from the cooling in the gas buffering and cooling vessel can be discharged from a condensed water valve 66 installed at the bottom of the gas buffering and cooling vessel 3.

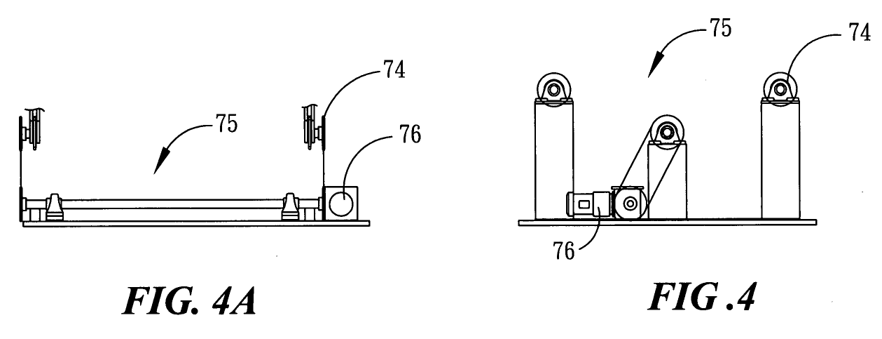

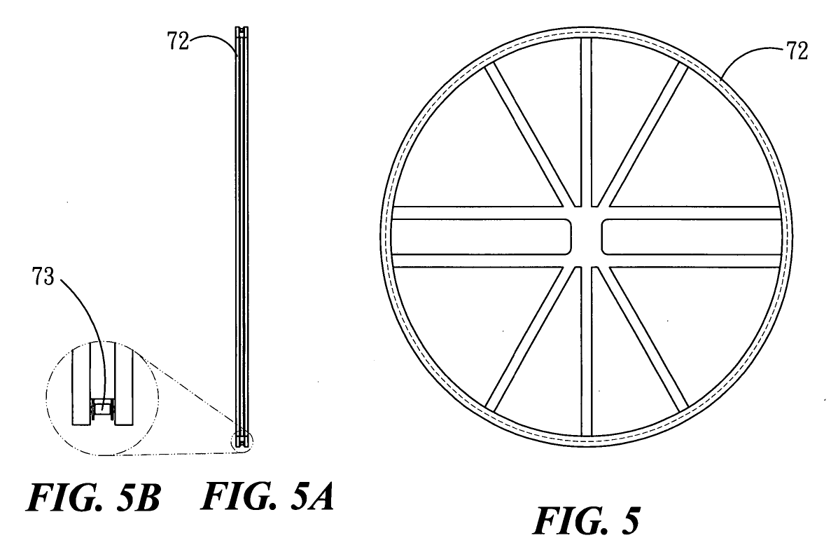

With reference to FIG. 1a, the driving system 7 consists of two supporting beams 71 for the waste-treating vessel 1. The two ends of each supporting beam 71 are connected to a reciprocating wheel 72, as shown in FIGS. 4 and 5. As shown in FIG. 5B, the wheel 72 is driven by a chain or gear 73, which is in turn driven by a driving gear 74 (FIG. 4A). The driving gear 74 is installed on the top of a supporting base 75 and driven by a motor 76 (FIG. 4). Further, the waste-treating vessel 1 in the apparatus according to the invention can be moved reciprocatingly using a rotary mode or other approaches to cut the medical waste reciprocatingly.

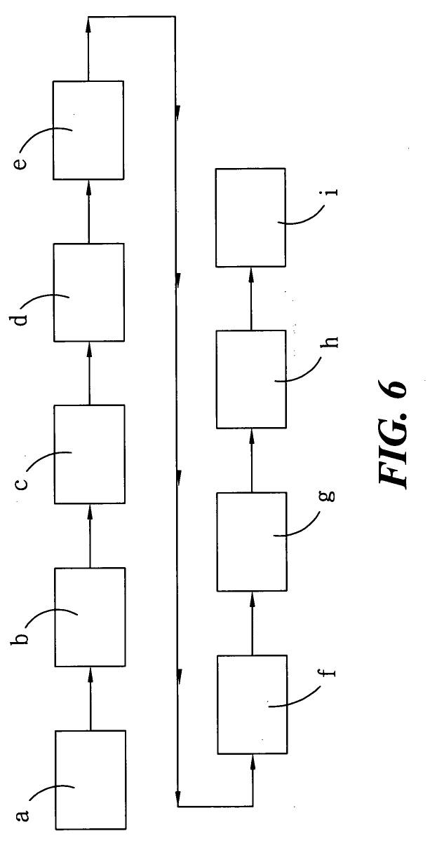

With reference to FIG. 6, the apparatus described above is operated by the following procedure:
a. Turn off all the valves of the gas buffering and cooling vessel 3, except for the outlet 46. Turn on the vacuum pump 41 to withdraw air from gas buffering and cooling vessel 3 to the atmosphere until a preset vacuum of the gas buffering and cooling vessel 3 is achieved;
b. Turn on the driving motor 76 of the driving system 7 to rotate the waste-treating vessel 1 at a preset angle convenient for waste loading. Open the closure member 11 and load the wastes (normally 50˜250 kg/batch) to the waste-treating vessel 1;
c. Close the closure member 11 and all the ports of the waste-treating vessel 1, except for the air inlet 44 of the gas buffering and cooling vessel 3 and the air outlet of the treatment vessel 1. Turn on the vacuum pump 41 to withdraw the air from the waste-treating vessel 1 into the gas buffering and cooling vessel 3 until a present vacuum of the waste-treating vessel 1 is achieved;
d. Open the steam inlet 54 and turn on the steam generator 51 to facilitate steam into waste-treating vessel 1 to increase the temperature and pressure within the feed charging and discharging chamber 13 (such as 135° C. and 3.4 atm);
e. Turn on the cutter and transmission system 2 and the driving system 7 so that the waste materials can be fractured into small pieces by moving back and forth between the feed charging and discharging chamber 13 and storage chamber 14.
f. After a period of time of treatment, open the air inlet 44 of the gas buffering and cooling vessel, the air outlet 47 of the waste-treating vessel, and the vacuum pump 41 to allow air being circulating between the gas buffering and cooling vessel 3 and the waste-treating vessel 1 for sterilization of the contaminated air withdrawn from the waste-treating vessel 1 for pre-vacuum. The steam valve 53 is remained open during the entire treatment of the waste.
g. After a preset period of time, turn off the cutter and transmission system 2 and the driving system 7. Turn on the cooling water circulation pump 61 and cooling device 62 to allow the steam in the waste-treating vessel 1 to be cooled off until a preset temperature and pressure.
h. Open the condensed water valve 8 to discharge the water condensed in the gas buffering and cooling vessel 3 as a result of the cooling process.
i. Turn on the driving system 7 so that the feed inlet and outlet 12 of the waste-treating vessel 1 is rotated to a position convenient for unloading of the waste being completely processed. The processed waste materials can now be further treated or disposed as the regular wastes.
   [Features and Effects]

The apparatus having a reciprocating autoclave with internal cutters (RAIC) for treatment of medical wastes provided according to the invention has following advantages over the recited patents and other conventional techniques:
1. With the inventive apparatus, medical wastes such as, for example, needles, surgical knife, syringes, rubber hose or tissue sections, and the like, could be placed directly in the treating vessel of the inventive apparatus without being sorted and screened, such that the labor and time can be saved and undue secondary contamination can be avoided.
2. The inventive apparatus can accomplish the treatment of medical wastes by crushing, sterilizing and cooling waste materials simultaneously in the treating vessel during a continuous treating process such that the steam penetratability can be increased and that the entire treatment can be completed at reduced time, comparing with conventional autoclaving methods with greatly increased efficiency.
3. The inventive apparatus is particularly designed to have a gas buffering and cooling vessel, whereby a pre-vacuum operation of the waste-treating vessel can be carried out to eliminate the formation of dead spaces among cold regions during steam sterilization and hence the treating efficiency could not be influenced.
4. With the inventive apparatus, gas drawn in the gas-buffering vessel during the pre-vacuum operation can be transferred continuously into the waste-treating vessel for sterilizing so that the subsequent treatment and disposal in the conventional technique can be eliminated.
5. After sterilization of the waste in the inventive apparatus, the high temperature steam in the waste-treating vessel can be transferred and cooled in the gas buffering and cooling vessel so as to avoid the emission of the mist and aerosols upon opening the cover of the vessel associated with the prior art technique and hence eliminate the problem relative to the view of odor.
6. The complete computerized operating process provided on the inventive apparatus can let the user accomplish the treatment of the medical waste material followed with a proper manner and simplified operating procedure without risk of operation accident.
7. The inventive apparatus is designed to be a compact profile and structure without the necessity of providing external track and crushing device so that necessary space for installing the apparatus can be greatly reduced and at the same time, both of the sterilization and crushing requirements can be achieved in a short time period and hence the sterilizing efficiency can be greatly increased and the cost of treating medical wastes can be lowered.

Many changes and modifications in the above described embodiments of the invention can, of course be carried out without departing from the scope thereof. Accordingly, to promote the progress in science and the useful arts, the invention is disclosed and is intended to be limited only by the scope of the appended claims.

What is claimed is:

1. A method for operating an apparatus for a crushing treatment of medical waste materials, the apparatus having a waste-treating vessel for holding the medical waste materials, a gas buffering and cooling vessel connecting to outside of the waste-treating vessel for cooling, and a cutter and transmission system for crushing the medical waste materials, the method comprising the steps of:
closing all valves of the gas buffering and cooling vessel, and withdrawing air from the gas buffering and cooling vessel until a preset vacuum is achieved;
turning the waste-treating vessel to a position for introducing the waste materials into the vessel;
closing a closure member and all valves, except for an air inlet and outlet of the waste-treating vessel and the gas buffering and cooling vessel, withdrawing air from the waste-treating vessel into the gas buffering and cooling vessel until the preset vacuum is achieved;
introducing steam into the waste-treating vessel until a preset high temperature and pressure is achieved;
turning on the cutter and transmission system and moving reciprocatingly the waste-treating vessel, thereby crushing the medical waste materials;
at the time of crushing the medical waste materials, opening the air inlet and outlet between the gas buffering and cooling vessel and the waste-treating vessel to allow air circulating therebetween for sterilization the contaminated air withdrawn from the waste-treating vessel and, optionally, introducing steam depending on the change of pressure and temperature in the waste-treating vessel;
stopping the cutter and transmission system and the vessel while closing a steam valve, turning on a cooling system inside the gas buffering and cooling vessel to lower the temperature and reducing the pressure in the waste-treating vessel by means of circulation of air between the vessel and the gas buffering and cooling vessel until the preset pressure is achieved across the waste-treating vessel and the gas buffering and cooling vessel;
discharging cooling water in the waste-treating vessel and the gas buffering and cooling vessel; and
turning the waste-treating vessel to a position, unloading treated medical waste materials, and carrying out further treatment and disposal as on a regular waste.

2. The method as in claim 1, wherein the waste-treating vessel moving reciprocatingly in a mode of rotation or swinging to crush adequately the medical waste material.

3. An apparatus for crushing treatment of medical waste materials, comprising:
a waste-treating vessel, provided with a feed inlet and outlet on one end and a closure member over the feed inlet and outlet, wherein the waste-treating vessel contains a feed charging and discharging chamber in the feed inlet and outlet, and a storage chamber located from the feed inlet and outlet to the other end thereof;
a cutter and transmission system installed within the waste-treating vessel and located between the feed inlet and out and the storage chamber, wherein the cutter and transmission system has a plurality of cutters for crushing the medical waste materials;
a gas buffering and cooling vessel, connecting to outside of the waste-treating vessel;
a vacuum system having a piping system and connecting the waste-treating vessel and the gas buffering and cooling vessel for withdrawing air from the gas buffering and cooling vessel to a predetermined vacuum, or for withdrawing air from the waste-treating vessel into the gas buffering and cooling vessel for carrying out a predetermined gas circulation;
a steam supply system for generating and introducing steam into the feed charging and discharging chamber; and
a heat exchange system installed within the gas buffering and cooling vessel for cooling hot air in the waste-treating vessel after completion of the treatment; and
a driving system for the waste-treating vessel, installed on a body of the waste-treating vessel to drive back and forth movement of the waste-treating vessel.

4. The apparatus as claimed in claim 3, wherein the feed charging and discharging chamber comprises 70-75 volume % of the waste-treating vessel, and the storage chamber comprises the remaindering volume % of the waste-treating vessel.

5. The apparatus as claimed in claim 3, wherein the vacuum system comprises a vacuum pump, a piping system, and corresponding electromagnetic vacuum valves on the piping system, wherein one end of the piping system extends to a position on the waste-treating vessel and the gas buffering and cooling vessel, and gas inlets are provided at positions between the piping system, the waste-treating vessel and the gas buffering and cooling vessel.

6. The apparatus claimed as in claim 3, wherein steam supply system comprises a steam generator, a piping system, and corresponding electromagnetic vacuum valves on the piping system, wherein one end of the piping system extends to a position on the waste-treating vessel, and steam inlets are provided at positions between the piping system and the waste-treating vessel.

7. The apparatus claimed as in claim 3, wherein the heat exchange system comprises a cooling water pump, a cooling device, a heat exchanger, a cooling water pipeline, and cooling water electromagnetic valves at the cooling water pipeline, wherein the heat exchanger is installed around the waste-treating vessel for introducing cooling medium through cooling water pipeline to carry out heat exchange.

8. The apparatus claimed as in claim 3, wherein the driving system comprises two horizontal beams provided on a surface of the waste-treating vessel, two reciprocating wheels fixed, respectively, to one end of the beams, two sets of chains or gears attaching on the wheels, two sets of driving gears installed on a supporting base and connected a driving motor which drives the reciprocating rotation of the chains or gears.

9. The apparatus claimed as in claim 3, wherein the cutter and transmission system comprises at least two cutter bases, a set of cutter shafts assembled in parallel of equal spacing on the cutter bases, a plurality of cutters mounted on one shaft alternatively docked into each spacing on the two neighboring shafts, and a motor for driving the rotation of each cutter shaft in a manner that cutters on the adjacent shaft engage with one another to give a function for crushing medical waste materials.

10. The apparatus claimed as in claim 3, wherein the cutter and transmission system comprises two or more sets of multiple-shaft cutter devices, and each cutter and transmission system comprises a plurality of cutters, wherein cutters near the feed charging and discharging chamber are bigger for the crude shredding of the medical waste materials and cutters near the storage chamber is smaller for fine grinding and so on, and wherein adjacent cutter and transmission systems are positioned to each other at a 90 degree angle.

11. The apparatus claimed as in claim 3, wherein the gas buffering and cooling vessel is provided to cover and seal over the outside of the feed charging and discharging chamber of the waste-treating vessel.
